(19) United States
(12) Patent Application Publication
(10) Pub. No.: US 2011/0106123 A1
(43) Pub. Date: May 5, 2011

(54) TWO-PART SURGICAL DEVICE

(76) Inventors: Michael LAFAUCI, Center Moriches, NY (US); Scott Berlin, Bayshore, NY (US); Russell Robertson, Brooklyn, NY (US); Jeanne Pfordresher, Brooklyn, NY (US); Stanley E. Mayer, Middle Town, NY (US)

(21) Appl. No.: 12/942,157

(22) Filed: Nov. 9, 2010

Related U.S. Application Data
(63) Continuation-in-part of application No. 12/557,186, filed on Sep. 10, 2009.
Application No. 12/557,186 is a continuation-in-part of application No. 12/025,909, filed on Feb. 5, 2008, now Pat. No. 7,818,885.

(51) Int. Cl. A61B 17/32

(52) U.S. Cl. 606/167

(57) ABSTRACT

A scalpel for performing complex surgeries, such as c-sections, is formed of a body that is preferably elongated and shaped and sized to fit ergonomically into the hands of surgeons. The body has surfaces that are joined to each other by smooth transition elements to insure that the body has no points or sharp edges that could cut or scratch the surgeon or the patient. The body is formed with a triangular notch formed of a beak and an extension. A surgical blade, preferably a standard off-the-shelf surgical blade, is imbedded in the body and the only portion of the blade that is accessible is a portion of its cutting edge spanning the notch. The scalpel is used by introducing the beak into a slit in the tissue with the blade portion facing the direction in which the cut is to be made. The body of the scalpel is then grasped firmly and advanced to make the cut. The initial incision can be made with the tip of the beak. In one embodiment, the body is molded around the blade. in another embodiment, the body is made of two parts that are welded together.

### RELATED APPLICATIONS

This application is a continuation-in-part of application Ser. No. 12/557,186 which is a continuation-in-part of U.S. application Ser. No. 12/025,909 filed on Feb. 5, 2008 which in turn claims priority to U.S. provisional application Ser. No. 60/886,191 filed on Feb. 5, 2007; all incorporated herein by reference.

### BACKGROUND OF THE INVENTION

1. Field of the Invention

This invention relates to devices for performing obstetric surgery, and more particularly to a novel device for performing incisions for a caesarian operation, as well as other similar surgical procedures or operations.

2. Description of the Prior Art

Many surgical procedures require one or more incisions to be made in the skin of a patient to provide access to the respective organ requiring active intervention. Additional incisions may be required in the internal tissues as well. For example, a caesarian section (c-section) is started by making an incision at the level of the pubic bone. Additional incisions are made in the walls of the uterus to allow a physician to remove the fetus. At the present, these incisions are made typically with a standard scalpel. However, this accepted technique has been found to be undesirable for several reasons. One reason is that it is hard to control the depth of the cut being made. if the incisions are too deep, they may result in injury to the fetus.

In addition, in the current method a physician uses the blade tip of the scalpel to make the initial cut in the uterine wall and then reverses the scalpel to make an additional puncture. The physician could get injured while he is reversing the scalpel. Moreover, during the actual cutting phase, the physician typically uses his fingers to guide the scalpel along the uterus wall. However, existing scalpels have many sharp edges and points that can injure the physician, the mother or the fetus while the incision is made.

A further disadvantage of existing scalpels is that they are fairly narrow, thin and slippery and ergonomically unfit to hold, especially with a gloved hand, especially after they are covered with body fluids. As a result they are not really suited for operations, such as c-sections, requiring complex and detailed manipulations.

What is needed is a small, light-weight device for performing c-sections or other complex surgeries that can be used safely and effectively by physicians.

### SUMMARY OF THE INVENTION

The present invention is a device for performing incisions associated with c-sections and other surgical procedures. it consists of a body which is sized and shaped so that it can be held securely and comfortably by a physician. It includes a body forming a beak used to make an initial puncture in the skin or other membrane. The beak is then inserted into the resulting hole and is advanced, causing the blade to make an incision of a desired length. As the incision is made, a bottom surface of the beak insures an adequate separation between the cutting blade and the underling tissue, thereby preventing undesirable damage to the physician, the mother or the fetus. Preferably, the scalpel is formed of a body having no sharp points or edges and having an ergonomic shape that facilitates different kinds of manipulations. The scalpel can be used for c-sections, laparoscopic procedures, etc.

In an advantageous embodiment, the body is formed of two unequal parts, with one part forming a cavity that houses the second part.

### BRIEF DESCRIPTION OF THE DRAWINGS

FIG. 1 is a side view of one embodiment of the cutting device constructed in accordance with this invention;

FIG. 2 is a perspective view of the cutting device of FIG. 1;

FIG. 3 is a cross-sectional view along line 3-3 of the cutting device of FIG. 2;

FIG. 4 is a side view of a first alternate embodiment of the invention;

FIG. 5a shows a side view of a second alternate embodiment of the invention;

FIG. 5b shows a front view of the embodiment of FIG. 5a;

FIG. 5c shows a top view of the embodiment of FIG. 5a;

FIG. 6a shows a side view of a third alternate embodiment of the invention;

FIG. 6b shows a front view of the embodiment of FIG. 6a;

FIG. 6c shows a top view of the embodiment of FIG. 6a;

FIG. 6d shows an enlarged top view of the beak area of the embodiment of FIG. 6a;

FIG. 6e shows an enlarged front view of the beak area of the embodiment of FIG. 6a;

FIGS. 6f-6i are cross-sectional views taken along lines f-f, g-g, h-h,i-i respectively in FIG. 6c; FIG. 6i also shows an alternate embodiment in which the scalpel body is made of two parts joined together by ultrasonic welding or other means;

FIG. 6j is a partial orthogonal view of the beak of the embodiment of FIG. 6a;

FIG. 7a shows a front view of the embodiment of FIG. 6a in a first position used to make the initial puncture for a surgery operation;

FIG. 7b shows a front view of the embodiment of FIG. 6a in a second position for starting the cut in the wall of the uterus;

FIG. 7c shows a front view of the embodiment of FIG. 6a in a third position for actual making the cut;

FIG. 7d shows the scalpel in the position of FIG. 7c further indicating the fingers of the physician; and

FIG. 8 shows a partial orthogonal view of another embodiment of the invention

FIG. 9 shows a plan view of another alternate embodiment of the invention

FIG. 10 shows an exploded view of the embodiment of FIG. 9; and

FIG. 11 shows a partial sectional view of yet another embodiment of the invention.

### DETAILED DESCRIPTION OF THE PREFERRED EMBODIMENTS

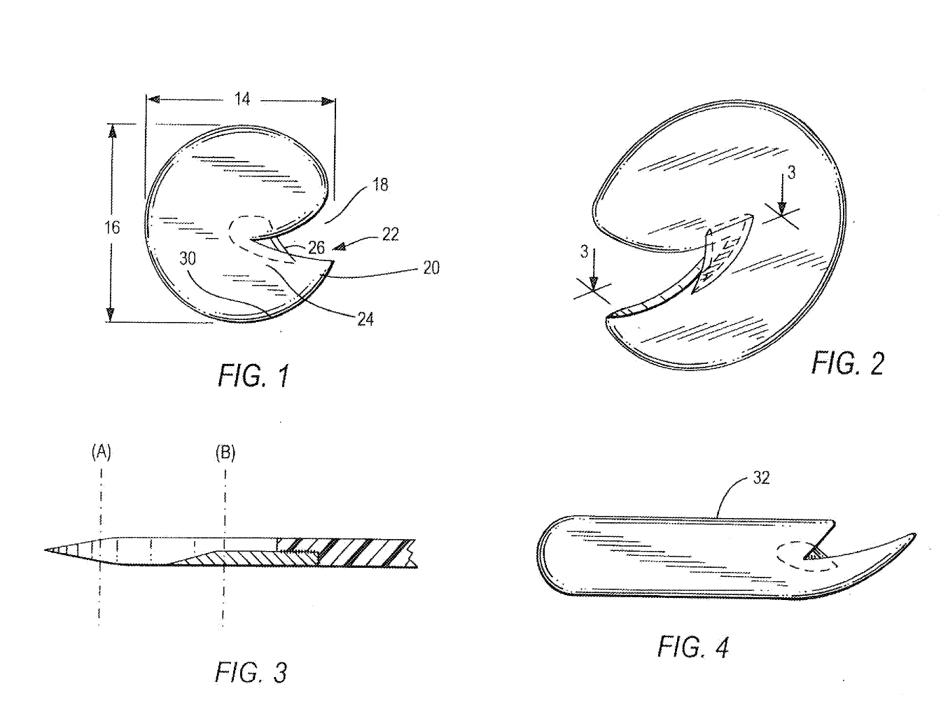

Referring first to FIGS. 1-3, a device 10 for performing incisions for a c-section includes a generally oval body 12 having a width 14 and a height 16 of about 50-80 mm and a thickness of about 3-6 mm. Other dimensions and shapes are suitable as well, as long as the device is light and comfortable to hold. The body is made with a triangular notch 18 forming a beak 20. The beak 20 is terminated with a rounded point 22. The notch holds a cutting blade 24 formed with a sharp edge 26.

The device 10 is used as follows. First, the tissue is punctured with the point 22. The beak 20 is then inserted through the resulting hole and advanced until the lateral side of the hole (not shown) is reached by the edge 26. The device is then advanced causing the edge 26 to make a clean incision of the desired length. While the skin or other membrane (e.g., the uterus) is being cut during this process, a lower surface 30 of the beak 20 separates the tissues of the organ or the fetus disposed immediately beneath and insures that a minimum spacing is maintained, thereby preventing any incidental damage. To ensure that device 10 cannot cut the physician, mother or fetus, surface 30 is either round or fiat. Moreover, the size and shape of the device insures that a physician does not prick or cut himself during surgery.

The device can be made from a single solid material. However, it is preferably to have the body 12 made of a suitable plastic material that is easy to make, using well known molding or other techniques, and is easy to sterilize. The cutting blade can be made of surgical steel or other similar materials and can be either imbedded in the body 12 during molding or can be attached later.

In FIG. 4, an alternate device 32 is shown with an elongated body. This shape may be preferred by some physicians because it is similar to a traditional scalpel and therefore may look more familiar.

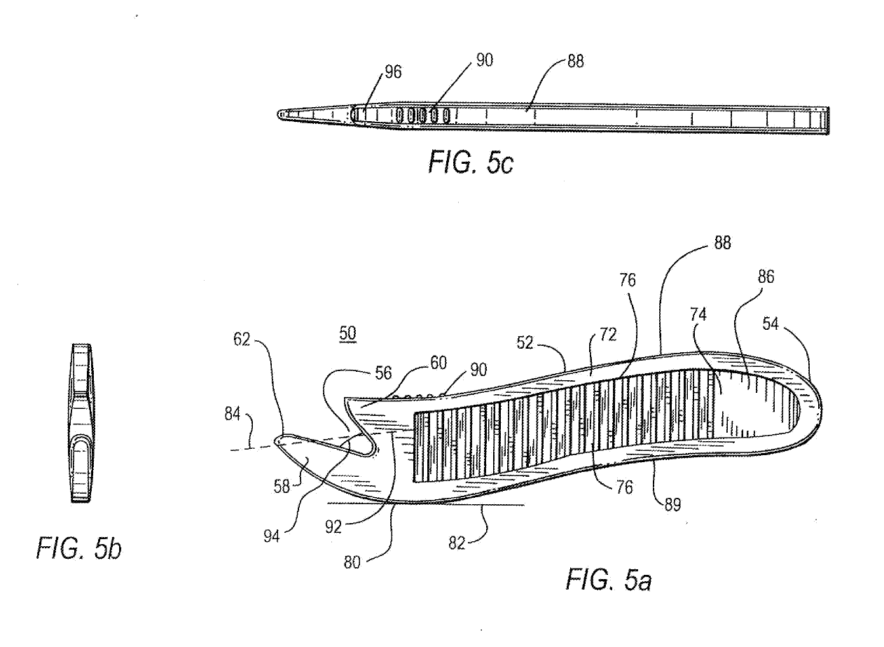

Studies were conducted with several physicians to ergonomically optimize the shape, size and configuration of the scalpel. Two such configurations are described below. FIGS. 5a-5c shows one such embodiment. In this embodiment, scalpel 50 an elongated curved body 52 that is rounded at one end 54 and a triangular notch 56 at the other end. The notch 56 is formed between the beak 58 and an extension 60. Beak 58 terminates in a rounded point 62. As seen in FIG. 5b, the beak 58 has a triangular cross-section thicker at the bottom than the top. This novel shape insures that the point 62 is strong and does not deflect when used to apply pressure and pierce a tissue or membrane of the patient as discussed above. The extension 60 is shorter then the beak 58 to provide good viewing angles of the blade and of the portion of tissue being cut.

The body 52 has two identical lateral surfaces, one such surface 70 being visible in FIG. 5a. This surface is formed with a peripheral rim 72 separated from a central depression 74 by a rounded ledge or rail 76. This rounded ledge or rail 76 can be used to easily grasp, push or pull the scalpel 50. Prior art scalpels do not possess any such features,

The central depression 74 is formed with a plurality of parallel vertical ridges 78. As seen in FIG. 5a, when the scalpel 50 is oriented so that its lowest point 80 is tangential to an imaginary horizontal surface 82, the ridges 78 are perpendicular to the horizontal surface 82. When the scalpel is used, the imaginary surface 82 is parallel to the tissue 84 being cut. Therefore, the ridges 78 provide the physician with an indication of how the scalpel 50 is oriented with respect to the tissue 84.

Preferably near the rounded end 54, the depression 74 has a flat or blank area 86 that can be used to provide a logo, a model number, etc.

Scalpel 50 has a top edge 88 that is formed near extension 60 with several protrusions 90. The protrusions 90 provide more friction when the physician has to apply pressure on edge 88 with a finger during a procedure. The top edge 88 and the bottom edge 89 form longitudinal edges for the scalpel 50 and are curved to provide the scalpel with a body that is easy to grasp. The curvature of the edges and the protrusions 90 provide the physician with a clear indication of the correct orientation for the scalpel 50 during surgery.

An important part of the scalpel 50 is blade 92. Preferably the body 52 is molded from a plastic material over the blade 92 and therefore in FIG. 5a only a portion 94 of the cutting edge of the blade is visible. This portion 94 can be straight, convex or concave. Returning to FIG. 5c, near its forward end, the edge 88 is formed with a knife indication 96 in the form of a line or depression corresponding to the position of the portion 94. Therefore the indication 96 assists the physician in determining the exact position of the cutting edge portion 94.

Preferably, the body 52 has an overall length of about 3-5 in, and more particularly between 4.800-5.00 in, because range can accommodate physicians with hands in the range of 6-8 in. The width of the body can range between 0.800-1.000 in and a thickness of between 0.150 and 0.250 in. The present inventors have found that a scalpel having a body of about 4.82 in in length, a width at point 80 of 0.916 in and a maximum thickness of 0.200 in is particularly advantageous.

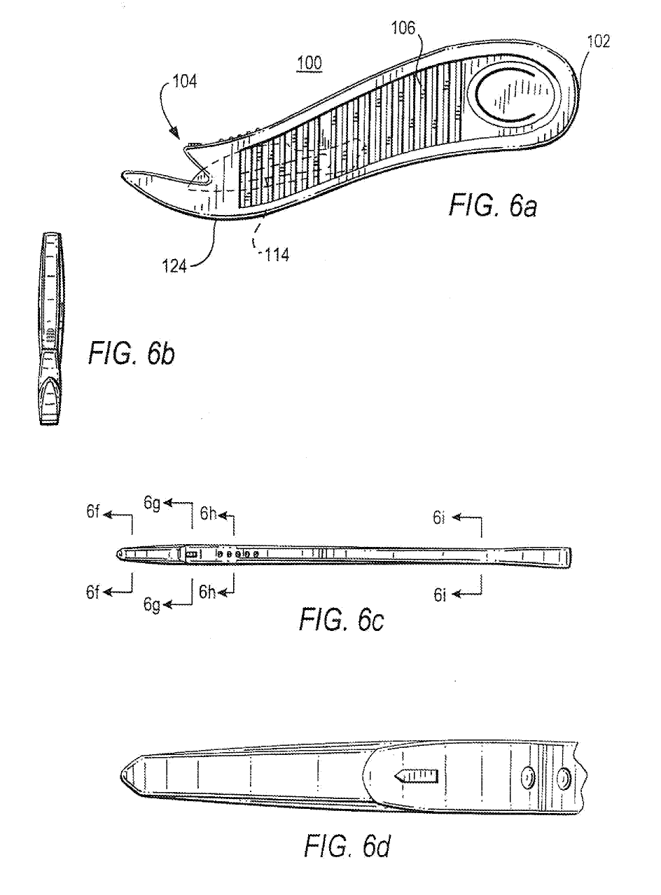

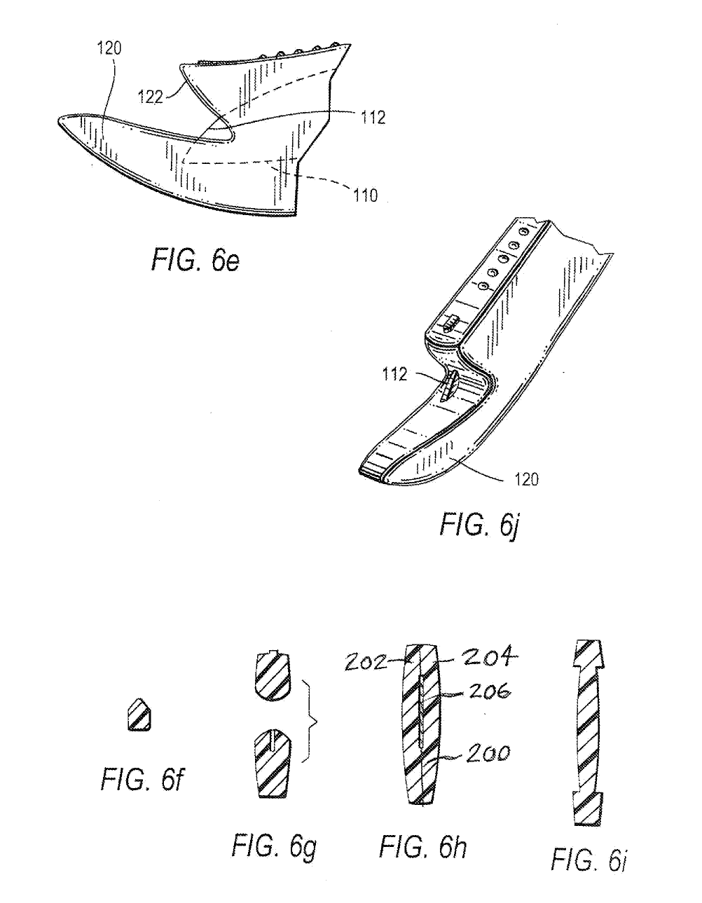

FIGS. 6a-6j display another embodiment of the invention similar to the one in FIGS. 5a-5c. In the embodiment just described, the inventive device has a somewhat uniform width, except at the forward end. In this latter embodiment, the scalpel 100 is tapered so that it is wider near the rounded end 102 and then near the notch 104. The scalpel 100 is also thicker. The preferred dimensions of the scalpel 100 are 4.863 in×0.916 in (at point 124)×0.250 in. Thus, overall this latter embodiment provides a heftier feel. Moreover, the extended width results in longer grooves 106 to accommodate hands of different sizes as well as more positions for the hands on the scalpel 100.

FIG. 6a also shows the outline of the blade 110 with its visible cutting edge portion 112. Preferably blade 110 is a standard surgical blade that is readily available from numerous sources. In the figure, blade 110 is a No. 23 blade with an elongated cutout 114. Other blades can be used as well. Alternatively, a custom designed blade may be used. Of course, such a blade may increase the cost of the scalpel.

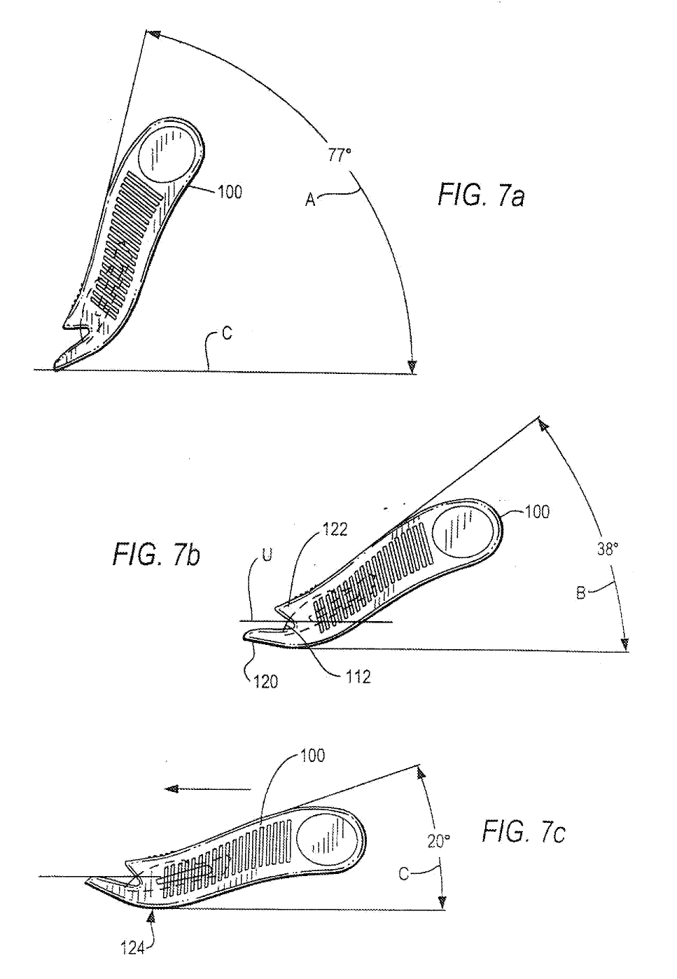

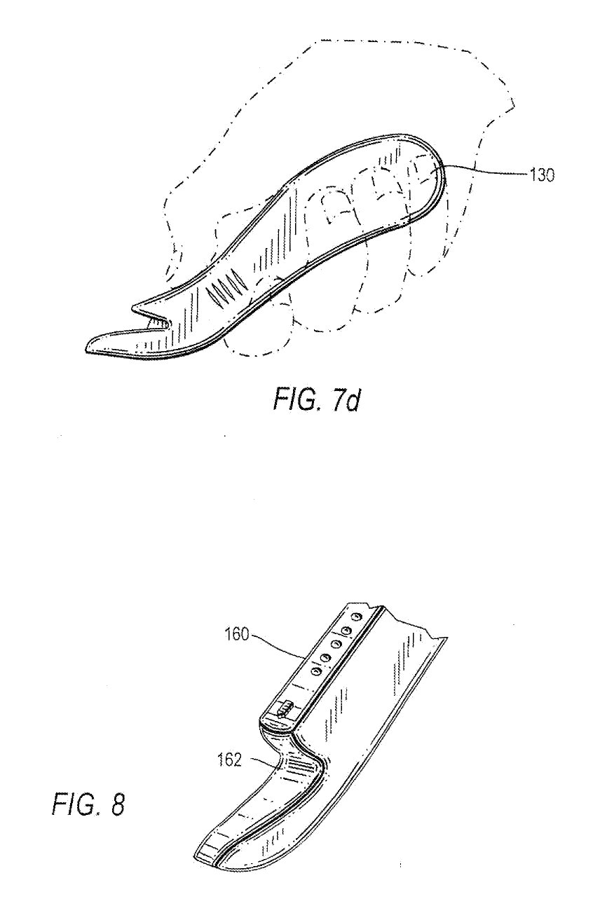

FIGS. 7a-7c illustrate how the embodiments of FIGS. 5a-c and 6a-d may be used to perform a c-section. In FIG. 7a the physician positions a scalpel (e.g. 100) as shown. The angle A may be in the range of 75-80 degrees and preferably about 77 degrees. The scalpel is used in this position to puncture the wall of the uterus U. If the wall is too thick, the physician may cut a small (1 in) opening therein with a standard scalpel. The beak 120 is then positioned under the wall U at an angle B in the range of 35-40 degrees and preferably 38 degrees. The scalpel 100 is designed so that it is comfortable to hold in this position. Because the extension 122 is shorter then the beak 122, the cutting edge 112 is visible to the physician from the top. The physician then rotates the scalpel 100 until it reaches an angle C of about 15-25 degrees and preferably 20 degrees, and starts advancing the scalpel. In this position, the beak 120 pushes the wall U slightly upward and ready to be cut by the edge 112. While this motion is continued, the sharp edge 112 cuts the wall U smoothly and the point 124 insures that the scalpel passes smoothly over the fetus and that the fetus is not injured. FIG. 7d shows the scalpel of FIG. 6c in the hands of the physician. The circles, e.g. circle 130, indicate the position of the physician's fingers during this step.

As discussed above, the body of the scalpel is overmolded on the standard surgical blade from a suitable plastic material. Except for the cutting edge of the blade, the body is formed with surfaces that are joined with rounded transitions with a nominal radius of about 0.030 in to insure that it presents no sharp edges or points. In this manner, the scalpel prevents injuring and infecting the physician, the patient and the fetus. The body can be provided in colors different from those of other instruments or in particular colors for different hospitals and departments to avoid confusion. The body can also be made of, or include, a compound visible by x-rays so that after the surgery is completed, the patient can be x-rayed to insure that the scalpel has not been left in the patient inadvertently. The device is made preferably of relatively cheap materials so that its manufacturing costs are reduced and so that it can be discarded, either after each use, or after each procedure.

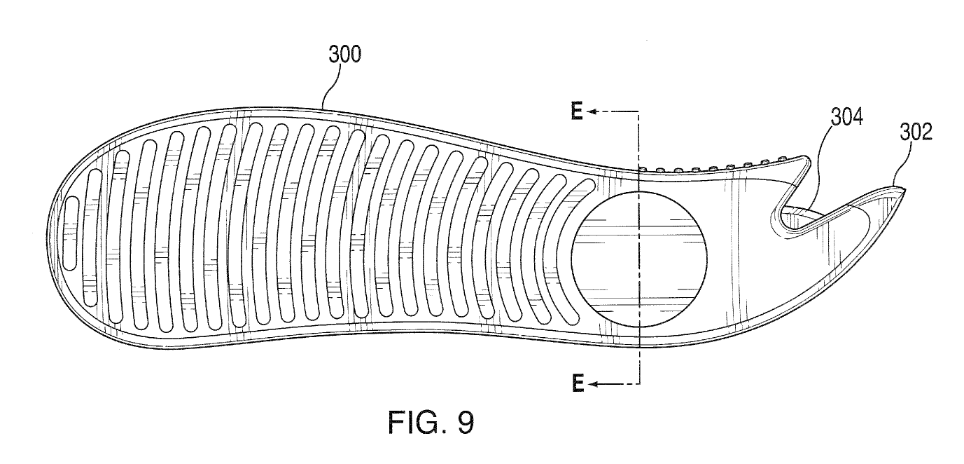

Another embodiment of the invention is shown in FIG. 8. In this embodiment a scalpel is shown having a body 160 similar to the ones in FIG. 5 or 6; however it does not have an imbedded steel blade. Instead, the base notch is shaped so that a plastic blade 162 is formed between the extension 164 and the beak 166. The blade has to be hard enough to be able to cut tissues, just as described above. The blade 162 is made integrally with the body 160 in a single molding operation.

In summary, the present invention presents a disposable surgical scalpel, such as an obstetrical scalpel, which aids in safer c-section deliveries by reducing knife injuries made from scalpel blades. Manufactured from a strong, break resistant plastic material, such as Lexan®, with a surgical blade embedded within, the scalpel provides enough resistance to pierce through the last tissues of uterine and amniotic tissue and cleanly cut across the uterus without lacerating the baby. Briefly, in the context of Caesarean section delivery, the factors that need to be taken into consideration when designing such a scalpel are: safety, performance, intuitiveness, and construction. The present scalpel has been designed with these factors in mind, as illustrated below. As a result, these factors provide a framework that renders the surgical scalpel very useful and unique.

Safety:
1. No knife lacerations made on baby due to unique piercing tip and embedded blade for a quick, clean incision.
2. Intuitive hand position increasing grip and control, decreases mis-use.
3. Clear sight lines over cutting edge.
4. Grip provides close proximity to blade to avoid loss of control while cutting.
5. conic shape, easily identifiable on the tool field.
6. Arrives to the Operating Room sterile, packaged, ready for surgery
7. Disposable post surgery

Performance:
1. Grip utilizes all fingers to increase control
2. Unique handle and blade configuration that pushes through the tissue like a scissor.
3. Ribbing perpendicular to cutting path increases grip.
4. Unique handle shape allows surgeon to back off or move up on tool, thereby increasing control and sight lines to cutting path.
5. Unique round edged tip will pierce through layers of tissue
6. Specific hand and tool position increase control and accuracy of incision.
7. Embedded surgical blade creates clean path through uterine tissues.
8. Increased sight-lines over cutting edge will decrease chance of vascular injury to mother.

Intuitiveness:
1. Iconic shape will not be confused for something else on the surgical tool field.
2. Grip discourages a downward piercing action, or an “ice pickin action due to it's unique grip and intuitive hand position
3. Tool can only be held two ways, pinched or grasped.
4. Clear sight lines provide a safe range of movement and motion through the uterine tissue.
5. Unique “jawin opening provides visual description of approximate tissue cutting depth.
6. Identifiable shape, won't be confused with another tool.

Construction:
1. Economical use of materials
2. Blade inserted into tool and cast into plastic C-Safe handle
3. Innovative break-resistant plastic
4. X-Ray opaque if lost of broken during surgery.

FIGS. 1-8 show some preferred embodiments of the invention. in yet another embodiment, instead of molding the body of the scalpel as a unitary single piece, the body is molded from two pieces with an indentation cored out to make a cavity from the blade. The two pieces can have a thickness of about 0.08 in at the handle area but smaller in the area corresponding to the blade.

After the two pieces 202, 204 are molded and cool off, the blade 206 is introduced into the cavity between the parts as shown in FIG. 6h and the parts are then placed together so they abut each other as shown in FIG. 6h with line 200 indicating the seam between the two parts. The two parts 202, 204 are then joined together permanently using ultrasonic welding or other well known means.

This construction is advantageous in that it is less expensive, provides faster cooling, and therefore reduces the length of time to make each scalpel. Moreover, the cavities in the two parts define more precisely the position of the blade 206,

In one embodiment, the two-part scalpel just discussed is made of a thermoplastic material, such as polyetherimide (PEI) available under the trade name of ULTEM® or other similar thermoplastic material.

Tests have shown that some doctors may prefer a scalpel made of a denser thermoplastic material then PEI so that it feels more solid. Therefore, in an alternate embodiment, the body of the scalpel can be made of acrylonitrile butadiene styrene (ABS) or other similar thermoplastic material. Preferably the ABS is impregnated with a suitable additive to render it radio opaque.

In yet another embodiment, the two parts 202, 204 are made with one or more additional indentations (not shown) that house a pellet made of lead or other similar relatively heavy material to make the scalpel heftier. Of course, in all the embodiments, the scalpel is made of a biocompatible material.

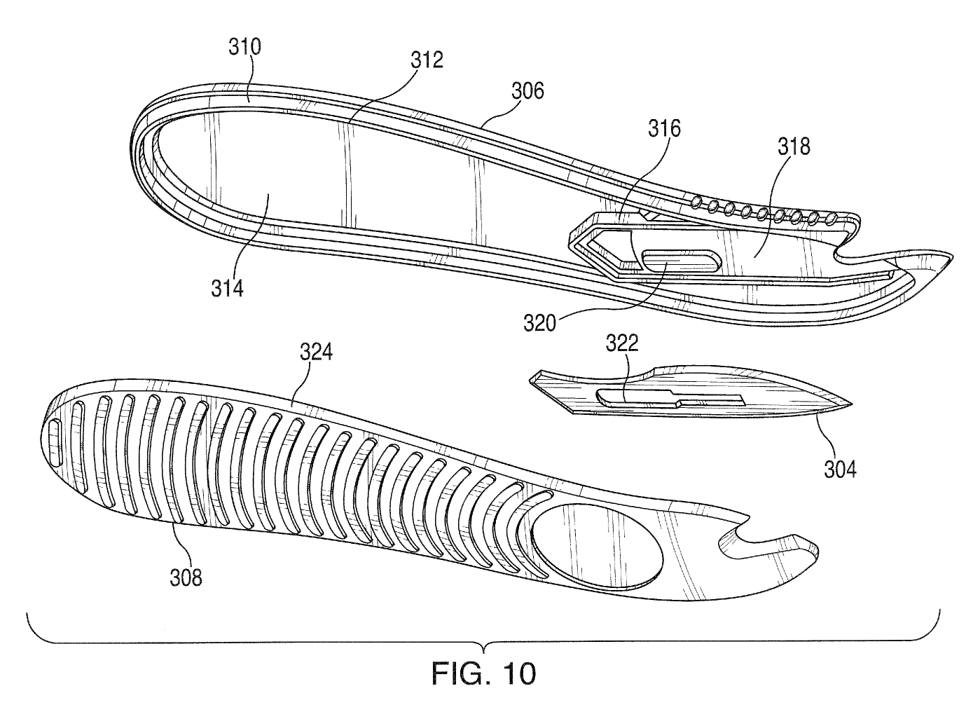

In the embodiment of FIGS. 9 and 10, a scalpel 300 is shown having a body with a beak 302 and a blade 304. As shown in FIG. 10, the body is formed of two parts 306, 308. Part 306 includes an outer peripheral wall 310 forming the entire outer edge of the body, and an inner wall 312 that defines an inner first cavity or depression 314. A second inner wall 318 is formed on the floor of the cavity 314 to form a second cavity 318. The second cavity 318 is formed with a buttress 320.

Blade 304 is formed with a standard cutout 322. The cavity 318 and buttress 320 are shaped to accept the blade 304 with the buttress 320 extending through the cutout 322.

Part 308 has an outer perimeter wall 324 sized and shaped to fit snugly into the cavity 314, with the was 312, 324 disposed adjacent to each other and with a portion of the part 308 covering the cavity 318 thereby capturing the blade 304. Once the part 308 is inserted into the cavity 314, the two parts 306, 308 are attached to each other by using sonic welding, an adhesive or other conventional means.

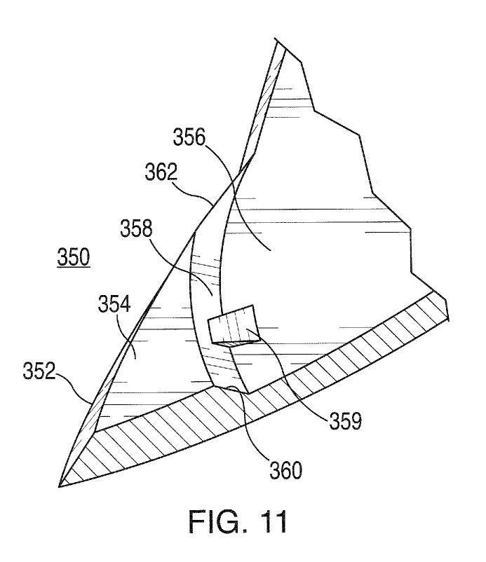

In FIG. 11, a partial view of a scalpel 350 is shown constructed in accordance with any of the embodiments disclosed above. Scalpel 350 is formed with a beak 352. The beak 352 is formed with an outer surface 354 that is coplanar or continuous with the rest of the side surface of the scalpel 350 and an inner surface 356. The two surfaces are joined by a wall 358. An intermediate ramp 360 is provided on the wall between its edges 362 and 364. This ramp 359 extends to approximately half the height of the wail 358 and is provided to reinforce the beak 352.

Because the base of the beak 352 connecting it to the rest of the scalpel has a thinner cross section at surface 356, the beak may have a tendency to bend or flex when a force is applied to it. The ramp 359 prevents the beak from bending or flexing.

Numerous modifications may be made to this invention without departing from its scope as defined in the appended claims.

We claim:

1. A scalpel comprising:
a plastic body having a flat shape with a periphery having two opposed longitudinal edges, one of said edges terminating in a triangular notch defining an extension and the other of said edges defining a beak, said beak being longer then said extension, said body being formed of a first and a second part with a first cavity being defined therebetween; and
a blade disposed in said first cavity and having a sharp edge portion extending between said extension and said beak and positioned to cut tissue passing between said beak and said extension.

2. The scalpel of claim 1 wherein said longitudinal edges are curved.

3. The scalpel of claim 1 wherein said body is formed with only rounded transitions between surfaces to eliminate sharp edges and points.

4. The scalpel of claim 1 wherein said first part includes an outer peripheral wall and an inner wall extending along said outer peripheral and defining a second cavity sized and wherein said second part includes an outer peripheral wall complementary to said inner wall to allow said second part to be nested within said second cavity.

5. A surgical scalpel comprising:
an elongated body shaped to fit comfortably in a hand and having two opposed flat surfaces disposed between two longitudinal edges, a round end and an another end formed with a notch formed by an extension and a beak; and
a surgical blade provided within the notch, said beak being arranged to direct flat tissue toward said cutting portion;
said elongated body being formed of two parts joined together to form said body.

6. The scalpel of claim 5 wherein said two longitudinal edges are curved to define a shape for holding in a palm.

7. The scalpel of claim 6 wherein said longitudinal edges define a substantially constant width for the body.

8. The scalpel of claim 7 wherein said longitudinal edges define a tapered width for the body.

9. The scalpel of claim 8 wherein said body is wider near the rounded end.

10. The scalpel of claim 5 further comprising grooves on said surfaces.

11. The scalpel of claim 10 wherein one of said longitudinal edges is a bottom edge having a bottom point disposed near said beak, wherein said grooves are perpendicular to a line tangential to said bottom point.

12. The scalpel of claim 5 wherein said surfaces are provided with grasping elements to allow the scalpel to be grasped during a surgery.

13. The scalpel of claim 5 wherein one of said longitudinal edges is a top edge, said top edge having protrusions.

14. The scalpel of claim 5 wherein one of said longitudinal edges has a marking aligned with said cutting portion to provide an indication of the position of said cutting portion.

15. The scalpel of claim 5 wherein body is defined by a plurality of surfaces, said surfaces being joined by rounded transitions to eliminate any sharp edges and points on said body.

16. The scalpel of claim 5 further comprising a surgical blade made of a metallic material and imbedded in said body, said blade having a cutting edge having a cutting portion that is exposed and spans said notch.

17. The scalpel of claim 16 wherein said blade is a standard surgical blade.

18. The scalpel of claim 5 wherein said extension is shorter than said beak to provide a clear line of sight to said blade as viewed from the top.

19. The scalpel of claim 5 wherein said beak has a triangular cross-section that is wider at the bottom than at the top supporting the blade, said beak terminating in a rounded point.

20. The scalpel of claim 19 wherein said parts include a first part and a second part, wherein said first part includes a first outer surface defining a beak surface for said beak, an inner surface that is formed at a distance inwardly of said first outer surface and a transition wall connecting said first outer surface and said inner surface.

21. The scalpel of claim 20 further comprising a ramp extending between said inner surface toward said first outer surface to reinforce said beak.

22. The scalpel of claim 21 wherein said ramp terminates on said intermediate wall.

23. The scalpel of claim 5 wherein said body is made of thermoplastic material.

24. The scalpel of claim 25 wherein said thermoplastic material is selected from the group consisting of PEI and ABS.
